(19) United States
(12) Patent Application Publication
(10) Pub. No.: US 2016/0235462 A1
(43) Pub. Date: Aug. 18, 2016

(54) System and Method for Plasma Sealing of Tissue

(71) Applicant: U.S. Patent Innovations LLC, Takoma Park, MD (US)

(72) Inventors: Jerome Canady, Lakeland, FL (US); Taisen Zhuang, Rockville, MD (US)

(21) Appl. No.: 15/042,180

(22) Filed: Feb. 12, 2016

Related U.S. Application Data
(60) Provisional application No. 62/115,405, filed on Feb. 12, 2015.

(51) Int. Cl. A61B 18/04; A61B 18/12

(52) U.S. Cl. CPC A61B 18/042; A61B 18/1206; A61B 2018/00595

(57) ABSTRACT

In a preferred embodiment, the present invention is a plasma sealer attachment for an electrosurgical system. The attachment has a connector assembly, a cable, and a hand piece. The hand piece has a splitter, a pair of tubes and a pair of conductive wires. When used, an inert gas flows through the connector assembly, down the cable and out both tubes. One of the conductive wires is connected to a source of monopolar electrosurgical energy and acts as an active electrode. The other conductive wire is connected to a neutral or ground and acts as a return electrode.

### CROSS-REFERENCE TO RELATED APPLICATIONS

The present application claims the benefit of the filing date of U.S. Provisional Patent Application Ser. No. 62/115,405 filed by the present inventors on Feb. 12, 2012.

The aforementioned provisional patent application is hereby incorporated by reference in its entirety.

### STATEMENT REGARDING FEDERALLY SPONSORED RESEARCH OR DEVELOPMENT

None.

### BACKGROUND OF THE INVENTION

1. Field of the Invention

The present invention relates to plasma devices and methods for treating tissues, and more specifically, to a system and method for monopolar sealing of tissue.

2. Brief Description Of The Related Art

The standard means for controlling traumatic and surgical blood loss are electrosurgical generators and lasers which respectively direct high-frequency electrical currents or light energy to localize heat in bleeding vessels so as to coagulate the overlying blood and vessel walls. Hemostasis and tissue destruction are of critical importance when removing abnormal tissue during surgery and therapeutic endoscopy. For monopolar electrosurgery electrical energy originates from an electrosurgical generator and is applied to target tissue via an active electrode that typically has a small cross-sectional surface-area to concentrate electrical energy at the surgical site. An inactive return electrode or patient plate that is large relative to the active electrode contacts the patient at a location remote from the surgical site to complete and electrical circuit through the tissue. For bipolar electrosurgery, a pair of active electrodes are used and electrical energy flows directly through the tissue between the two active electrodes.

A method of monopolar electrosurgery via argon plasma technology was described in U.S. Pat. No. 4,040,426 to Morrison and in U.S. Pat. No. 4,781,175 to McGreevy. This method, referred to as argon plasma coagulation (APC) or argon beam coagulation (ABC), is a non-contact monopolar thermoablative method of electrocoagulation that has been widely used in surgery for the last twenty years. In general, APC involves supplying an ionizable gas such as argon past the active electrode to target tissue and conducting electrical energy to the target tissue in ionized pathways as non-arcing diffuse current.

APC has been demonstrated to be effective in the coagulation of blood vessels and human tissue during surgery. APC functions in a noncontact manner. The electrical current is initiated only when the tip of the handpiece or catheter is within one centimeter of the target tissue and produces a homogenous 1 mm to 2 mm well-delineated eschar. The eschar created by APC is further characterized by a decrease absence of charring and carbonization compare to eschar resulting from conventional electrosurgical fulguration. The eschar remains firmly attached to the tissue, in contrast to other coagulation modalities where there is an overlying charred layer of coagulated blood. There is minimal tissue necrosis with APC.

Advances in the APC field have allowed to use of bipolar instruments that do not require the patient to be part of the electrical circuit because the instrument contains both source and return electrodes and the plasma is formed directly between the electrodes. In bipolar APC no current is conducted through the patient. For example, U.S. Pat. No. 7,549,990 and U.S. Pat. No. 7,122,035 disclose bipolar systems for use with argon plasma coagulation. In a typical bipolar APC system, the instrument includes a pair of electrodes across which a high potential is applied to ionize a flow of argon gas.

Monopolar APC systems typically use a standard electrosurgical generator as their power source. Such a standard electrosurgical generator will produce high voltage high frequency AC power and can be used with many different types of instruments in many different procedures. In contrast, bipolar APC systems commonly use dedicated high-voltage DC power generators.

U.S. Patent Application Publication No. 2014/0228833 to Friedrichs discloses a system and method for hybrid polarized/non-polarized plasma beam coagulation for variable tissue effects in which the inclusion of the patient in the electrical circuit can be varied.

### SUMMARY OF THE INVENTION

In a preferred embodiment, the present invention is a system for sealing vessels. The system has a connector assembly, a cable assembly, a hand piece and first and second conductive wires. The connector assembly has a fluid connector for connecting to a source of inert gas, a first electrical connector for connecting a wire to a source of monopolar electrosurgical energy and a second electrical connector for connecting to a ground. The hand piece has a splitter and first and second tubes. The first conductive wire has a distal end connected to said first electrical connector, a first portion extending through said cable assembly, a second portion extending through said splitter and a third portion extending into and to at least within 1 cm of a distal end of said first tube. The second conductive wire has a distal end connected to said second electrical connector, a first portion extending through said cable assembly, a second portion extending through said splitter and a third portion extending into and to at least within 1 cm of a distal end of said second tube. The system additionally may comprise a monopolar electrosurgical generator connected to said connector assembly and a source of inert gas connected to said connector assembly.

In another embodiment, the present invention is a system for sealing vessels. The system has a connector assembly comprising a fluid connector and first and second electrical connectors, a cable assembly having a proximal end connected to said connector assembly, said cable assembly having within it an elongated active electrode having a proximal end connected to said first electrical connector, an elongated ground electrode connected to said second electrical connector and a first channel for receiving flowing gas, wherein said active electrode and said ground electrode are insulated from one another in said cable assembly, a splitter connected to a distal end of said cable assembly for splitting said channel into second and third channels with said active electrode extending into and through said second channel and said ground electrode extending into and through said third channel, a first tube having a proximal end connected to said second channel, wherein said active electrode extends out of said second channel, into said first tube and through said first tube at least to within a centimeter of a distal end of said first tube, and a second tube having a proximal end connected to said third channel, wherein said ground electrode extends out of said third channel, into said second tube and through said second tube at least to within a centimeter of a distal end of said second tube.

In yet another embodiment, the present invention is a system for sealing vessels having a fluid connector, a fluid splitter having an entry port connected to said fluid connector and having first and second exit ports, a first channel connected to said first exit port, a second channel connected to said second exit port, a first electrical connector for connecting an active electrode to a source of monopolar electrosurgical energy, a second electrical connector for connected a ground electrode to a ground, an elongated active electrode and an elongated ground electrode. The elongated active electrode has a proximal end connected to said first electoral connector, an insulated portion extending from said first electrical connector at least to said entry port of said fluid splitter and an uninsulated portion extending through at least a portion of said first channel. The elongated ground electrode comprises a proximal end connected to said second electoral connector, a first portion extending from said second electrical connector at least to said entry port of said fluid splitter and an uninsulated portion extending through at least a portion of said first channel. The first portion of the elongated ground electrode may or may not be insulated.

Still other aspects, features, and advantages of the present invention are readily apparent from the following detailed description, simply by illustrating a preferable embodiments and implementations. The present invention is also capable of other and different embodiments and its several details can be modified in various obvious respects, all without departing from the spirit and scope of the present invention. Accordingly, the drawings and descriptions are to be regarded as illustrative in nature, and not as restrictive. Additional objects and advantages of the invention will be set forth in part in the description which follows and in part will be obvious from the description, or may be learned by practice of the invention.

### BRIEF DESCRIPTION OF THE DRAWINGS

For a more complete understanding of the present invention and the advantages thereof, reference is now made to the following description and the accompanying drawings, in which:

FIG. 1 is a perspective view of a plasma sealer attachment for a monopolar electrosurgical generator in accordance with a preferred embodiment of the present invention.

FIG. 2 is a diagram of a hand piece of a plasma sealer attachment for a monopolar electrosurgical generator in accordance with a preferred embodiment of the present invention.

FIG. 3 is a diagram of a connector assembly of a plasma sealer attachment for a monopolar electrosurgical generator in accordance with a preferred embodiment of the present invention.

### DETAILED DESCRIPTION OF THE PREFERRED EMBODIMENTS

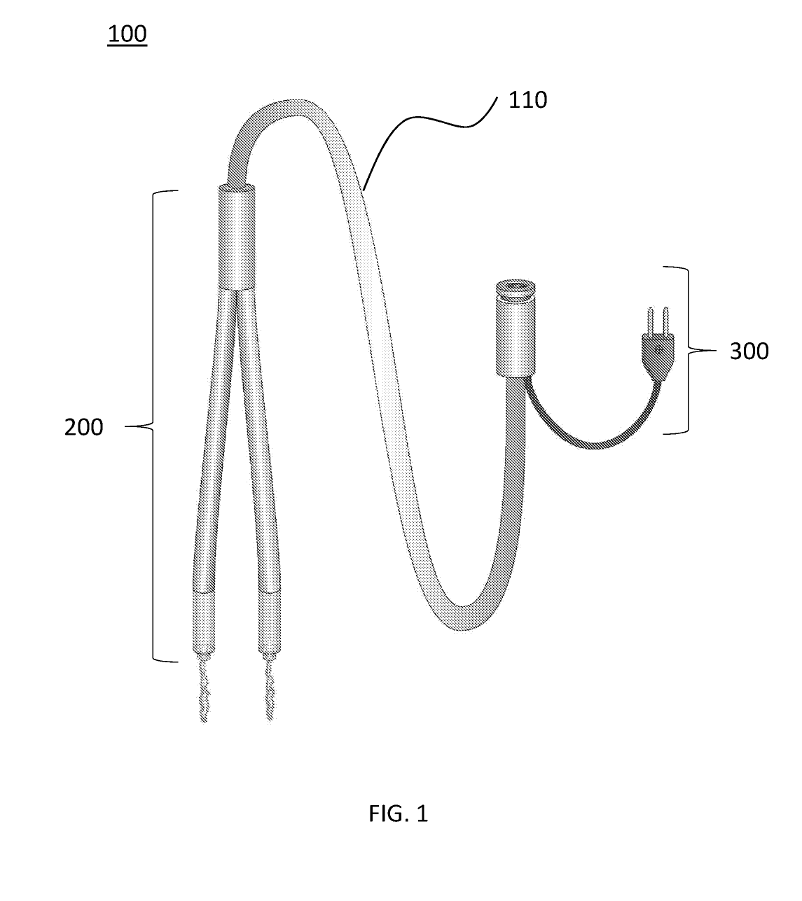

The preferred embodiments of the inventions are described with reference to the drawings. A plasma sealer attachment 100 for a monopolar electrosurgical generator in accordance with a preferred embodiment of the present invention is shown in FIG. 1. A plasma sealer hand piece 200 is connected to a connector assembly 300 by a multi-function cable 110. The cable 110 has a channel within it through which a gas flow from the connector assembly 300 to the hand piece 200. The cable 110 further has wires within it for transmitting electrosurgical energy from and to the connector assembly 300. The channel within the cable 110 may be a separate tube or may simple be the area within the cable around the wires. The wires within the cable, or cable assembly, are insulated from one another, for example, by one or both wires having an insulator around them. In alternate embodiments a bundle may be used rather than a multi-function cable.

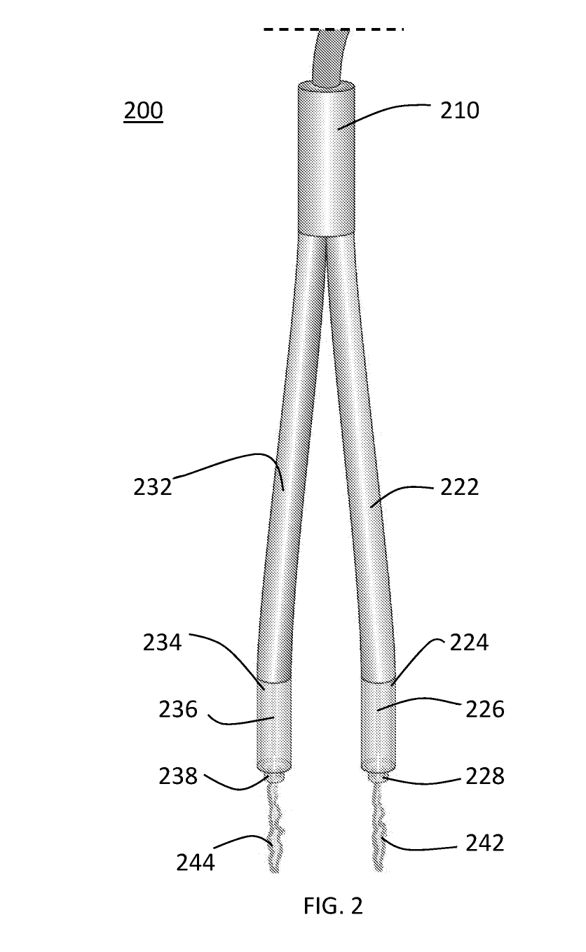

The plasma sealer hand piece 200, shown in FIG. 2, a splitter 210 for splitting the gas channel from the cable 110 to into channels within tubes 222 and 232. The splitter 210 further provides for conductive wires 226 and 236 to extend down tubes 222 and 232, respective. In other embodiments the hand piece can be eliminated and a splitter used in its place. In yet other embodiments, the splitter could be connected directly to the electrosurgical generator and the cable assembly could be eliminated.

As described below, the conductive wire 226 connects through the connector assembly 300 to a source of electrosurgical energy and acts as an active electrode. The conductive wire 236 connects through connector assembly 300 to a neutral or ground and acts as a return electrode. The tubes 224 and 234 may be elastic or may be bendable to permit a user to direct the flow of gas from the tube in various directions. The tubes 224, 234 may have tips formed from a non-stick, non-conductive high melting point material such as a ceramic material or PTFE. The tips 224, 234 may have a reduced diameter portion 228, 238, respectively, for increasing the flow velocity of gas exiting the tubes. The reduced diameter portions 228, 238 may be formed integrally with the tips 224, 234 or may be separate parts attached to, such as by insertion into, tips 224, 234.

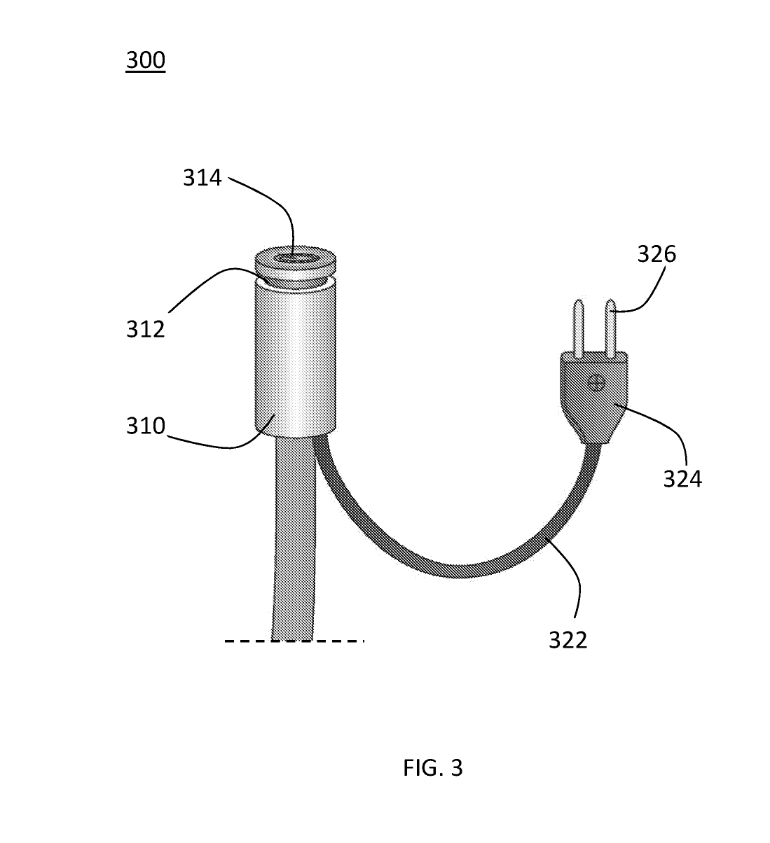

The connector assembly 300, shown in FIG. 3, has an insulator member 310, which may serve as a handle to permit a user to plug the connector assembly 300 into an electrosurgical generator, argon unit, or connecting cable (not shown). The insulator member 310 has a sealing area 312, such as a groove or lip for receiving a gasket. The insulating member 312 further has within it a metal connector 314 for delivering energy from an electrosurgical generator to the conductive wire 226. The metal connector 314 has a center hole or opening 316 through which gas flows into the channel in cable 110. The connector assembly 300 further has a flexible cable 322 extending to an insulator or handle 324 which has a plug or metal connector 326 for connecting to a neutral or ground. The flexible cable 322 has within it a conductive wire connected through the connector assembly to the conductive wire 226.

When in use, target tissue is placed generally between or in front of the tips 224 and 234. An inert gas such as argon flows from, for example, an argon unit through the opening 316 in metal connector 314, through a channel in the connector assembly 300 into the channel within cable 110. The inert gas flows through the channel in cable 110 to splitter 210 and into tubes 222 and 232. The inert gas flow out of both tip 224 and tip 234. Electrical energy supplied by a monopolar electrosurgical generator is delivered through metal connector 314 to the wire 226. When electrosurgical energy is applied to the wire 226, a plasma beam forms through the inert gas from the tip 224 to the target tissue and from the target tissue to the wire 236. Ground wire 236 effectively takes the place of a grounding pad that typically would be used with a monopolar electrosurgical system.

While the embodiment shown in FIGS. 1-3 is directed to open surgery, it will be apparent to those of skill in the art that alternate embodiments of invention for use in micro-invasive procedures such as laparoscopy, endoscopy and thorascopy, bronchoscopy and cystoscopy also are possible.

The foregoing description of the preferred embodiment of the invention has been presented for purposes of illustration and description. It is not intended to be exhaustive or to limit the invention to the precise form disclosed, and modifications and variations are possible in light of the above teachings or may be acquired from practice of the invention. The embodiment was chosen and described in order to explain the principles of the invention and its practical application to enable one skilled in the art to utilize the invention in various embodiments as are suited to the particular use contemplated. It is intended that the scope of the invention be defined by the claims appended hereto, and their equivalents. The entirety of each of the aforementioned documents is incorporated by reference herein.

What is claimed is:

1. A system for sealing vessels comprising:
a connector assembly having a fluid connector for connecting to a source of inert gas, a first electrical connector for connecting a wire to a source of monopolar electrosurgical energy and a second electrical connector for connecting to a ground;
a cable assembly; and
a hand piece, said hand piece comprising:
a splitter;
first and second tubes; and
a first conductive wire having a distal end connected to said first electrical connector, a first portion extending through said cable assembly, a second portion extending through said splitter and a third portion extending into and to at least within 1 cm of a distal end of said first tube;
a second conductive wire having a distal end connected to said second electrical connector, a first portion extending through said cable assembly, a second portion extending through said splitter and a third portion extending into and to at least within 1 cm of a distal end of said second tube.

2. A system for sealing vessels according to claim 3, further comprising a source of inert gas connected to said connector assembly.

3. A system for sealing vessels according to claim 1, further comprising a monopolar electrosurgical generator connected to said connector assembly.

4. A system for sealing vessels according to claim 3, further comprising a source of inert gas connected to said connector assembly.

5. A system for sealing vessels comprising:
a connector assembly comprising a fluid connector and first and second electrical connectors;
a cable assembly having a proximal end connected to said connector assembly, said cable assembly having within it an elongated active electrode having a proximal end connected to said first electrical connector, an elongated ground electrode connected to said second electrical connector and a first channel for receiving flowing gas, wherein said active electrode and said ground electrode are insulated from one another in said cable assembly;
a splitter connected to a distal end of said cable assembly for splitting said channel into second and third channels with said active electrode extending into and through said second channel and said ground electrode extending into and through said third channel;
a first tube having a proximal end connected to said second channel, wherein said active electrode extends out of said second channel, into said first tube and through said first tube at least to within a centimeter of a distal end of said first tube; and
a second tube having a proximal end connected to said third channel, wherein said ground electrode extends out of said third channel, into said second tube and through said second tube at least to within a centimeter of a distal end of said second tube.

6. A system for sealing vessels comprising:
a fluid connector;
a fluid splitter having an entry port connected to said fluid connector and having first and second exit ports;
a first channel connected to said first exit port;
a second channel connected to said second exit port;
a first electrical connector for connecting an active electrode to a source of monopolar electrosurgical energy;
an elongated active electrode comprising:
a proximal end connected to said first electoral connector;
an insulated portion extending from said first electrical connector at least to said entry port of said fluid splitter; and
an uninsulated portion extending through at least a portion of said first channel;
a second electrical connector for connecting a ground electrode to a ground; and
an elongated ground electrode comprising:
a proximal end connected to said second electoral connector;
a first portion extending from said second electrical connector at least to said entry port of said fluid splitter; and
an uninsulated portion extending through at least a portion of said first channel.

7. A system for sealing vessels according to claim 6, wherein said first portion of said elongated ground electrode is insulated.
